(19) United States
(12) Patent Application Publication
(10) Pub. No.: US 2013/0046019 A1
(43) Pub. Date: Feb. 21, 2013

(54) Methods of Treatment Using 3-Bromopyruvate and Other Selective Inhibitors of ATP Production

(71) Applicant: PRESCIENCE LABS, LLC, Wilmington, DE (US)

(72) Inventors: Jean-Francois Geschwind, Potomac, MD (US); Mustafa Vali, Baltimore, MD (US)

(73) Assignee: PRESCIENCE LABS, LLC, Wilmington, DE (US)

(21) Appl. No.: 13/658,696

(22) Filed: Oct. 23, 2012

Related U.S. Application Data
(63) Continuation of application No. 13/639,088.
(60) Provisional application No. 61/321,470, filed on Apr. 6, 2010.

(51) Int. Cl. A61K 31/19; A61P 31/04; A61P 31/10; A61P 17/02; A61P 33/00; A61P 35/00; A61P 29/00; A61P 31/00; A61P 31/12

(52) U.S. Cl. 514/557

(57) ABSTRACT

The present invention provides methods for preventing or treating infections, especially bacterial infections, by administering selective inhibitors of ATP production to inhibit glycolytic enzymes, such as glyceraldehyde 3-phosphate dehydrogenase (GAPDH). As such, the present invention further provides methods for preventing or treating inflammation and sepsis associated with infections, as well as increased wound healing and decreased wound scarring.

### CROSS-REFERENCE TO RELATED APPLICATIONS

This application is a continuation application of U.S. application Ser. No. 13/639,088, now pending; which is a 35 USC §371 National Stage application of International Application No. PCT/US2011/031458 filed Apr. 6, 2011, now expired; which claims the benefit under 35 USC §119(e) to U.S. application Serial No. 61/321,470 filed Apr. 6, 2010, now expired.

### BACKGROUND OF THE INVENTION

1. Field of the Invention

This invention relates generally to microbiology, and more specifically to treating microbial infections, especially bacterial infections, by selectively inhibiting the glycolytic enzymes, such as glyceraldehyde 3-phosphate dehydrogenase (GAPDH).

2. Background Information

The discovery and use of antibiotics has been one of the great achievements of modern medicine. Current sales of antibiotics are over $30 billion (U.S.D.) worldwide. Without antibiotics, physicians would be unable to perform complex surgery, chemotherapy or most medical interventions such as catheterization. However, the overuse and sometimes unwarranted use of antibiotics have resulted in the evolution of new antibiotic-resistant strains of pathogens, most notably bacteria.

Over the past several decades, the frequency of antimicrobial resistance and its association with serious infectious diseases have increased at alarming rates. Increasing use of antimicrobials in humans, animals, and agriculture has resulted in many microbes developing resistance to current antimicrobial drugs. The increasing prevalence of resistance among nosocomial pathogens is particularly alarming. Of the over 2 million nosocomial infections occurring each year in the United States, 50 to 60% are caused by antimicrobial-resistant strains of bacteria. This high rate of resistance increases the morbidity, mortality, and costs associated with nosocomial infections. In the United States, between 5 and 10 percent of all hospital patients develop an infections leading to an increase of about $5 to $10 billion in annual U.S. healthcare costs. About 90,000 of these patients die each year as a result of their infection, up from 13,300 patient deaths in 1992. Additionally, people infected with antimicrobial-resistant organisms are more likely to have longer hospital stays and often require more complicated treatment.

Particularly significant resistant bacterial types are represented by strains such as Staphylococcus aureus, pneumococci, enterococci, Klebsiella pneumoniae, Escherichia coli, and Proteus mirabilis, Enterobacter species, Citrobacter freundii, Pseudomonas aeruginosa, Acinetobacter, and Stenotrophomonas maltophilia. Antibacterial resistance is exaggerated by the increasing existence of bacterial strains resistant to multiple antibacterials. For example, some Pseudomonas aeruginosa isolates are virtually resistant to all antibacterials.

In addition to antibacterial resistance, many conventional antibacterials cause toxic side effects to normal tissue of subjects upon administration. With the dramatic rise of antibiotic resistance, including the emergence of untreatable infections, and toxicity concerns, there is a clear unmet medical need for new types of antibacterials, particularly antibacterials with novel mechanisms of action effective against all types of bacteria, including both gram-positive and gram-negative bacteria.

### SUMMARY OF THE INVENTION

The present invention is based in part on the seminal discovery that antiglycolitic compounds can kill and prevent the growth of microbes. Such compounds selectively inhibit glycolytic enzymes, such as glyceraldehyde 3-phosphate dehydrogenase (GAPDH), causing a depletion in ATP and death of the targeted organism.

Accordingly, the present invention provides a method of preventing or treating microbial infection in a subject in need thereof. The method includes administering to the subject an antiglycolytic halopyruvate, for example 3-bromopyruvate, thereby preventing or treating bacterial infection in the subject.

In another aspect, the invention provides a method of preventing or treating inflammation resulting from microbial infection in a subject in need thereof. The method includes administering to the subject an antiglycolytic halopyruvate, for example 3-bromopyruvate, thereby preventing or treating inflammation in the subject.

In another aspect, the invention provides a method of preventing or treating fever resulting from microbial infection in a subject in need thereof. The method includes administering to the subject an antiglycolytic halopyruvate, for example 3-bromopyruvate, thereby preventing or treating fever in the subject.

In another aspect, the invention provides a method of preventing or treating sepsis resulting from bacterial infection in a subject in need thereof. The method includes administering to the subject an antiglycolytic halopyruvate, for example 3-bromopyruvate, thereby preventing or treating sepsis in the subject.

In another aspect, the invention provides a method of increasing wound healing in a subject in need thereof. The method includes administering to the subject an antiglycolytic halopyruvate, for example 3-bromopyruvate, thereby increasing wound healing in the subject.

In another aspect, the invention provides a method of preventing or reducing wound scarring a subject in need thereof. The method includes administering to the subject an antiglycolytic halopyruvate, for example 3-bromopyruvate, thereby preventing or reducing wound scarring in the subject.

In another aspect, the invention provides a method of inhibiting the growth of a microbe. The method includes contacting the microbe with an antiglycolytic halopyruvate, for example 3-bromopyruvate, thereby inhibiting growth of the microbe. In certain embodiments, the microbe is contacted in vivo or in vitro.

In various embodiments, methods of the invention include administration of 3-bromopyruvate to prevent or treat microbial infection or manifestations of microbial infection, such as inflammation, fever, sepsis, wound scarring, decreased wound healing; and inhibiting or preventing growth of microbes by contact with an antiglycolytic halopyruvate, for example 3-bromopyruvate. In such embodiments, microbes include microorganisms, such as bacteria, parasites and fungi. In exemplary embodiments, the microbe is a bacteria and may be gram-negative, gram-positive, aerobic or anaerobic. Further, in various embodiments, an antiglycolytic halopyruvate, for example 3-bromopyruvate, may be administered with one or more agents, such as another antibiotic, antifungal, antiviral, antiparasitic, or chemotherapeutic.

### DETAILED DESCRIPTION OF THE INVENTION

The present invention provides methods using antiglycolitic compounds which act to prevent glycolysis in microbes by targeting key enzymes responsible for ATP generation. Antiglycolytic halopyruvates, for example 3-bromopyruvate, represent a new class of antibiotic that selectively inhibits glycolytic enzymes causing a depletion in ATP and death of the organism. Blocking ATP production renders microbes such as bacteria unable to perform functions including, but not limited to, biosynthesis, replication, motility, resistance and defense against host immune systems.

Before the present composition, methods, and culturing methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

Many microbes, such as bacteria, parasites and fungi, share common glycolytic pathways and therefore common glycolytic enzymes. For example, glyceraldehyde 3-phosphate, dehydrogenase (GAPDH) is conserved across bacteria, parasites and fungi. Similarly, different types of bacteria also share common glycolytic pathways and therefore common glycolytic enzymes since all bacteria use sugar metabolism to survive, regardless of whether the bacteria is gram-negative or gram-positive, or whether the bacteria is anaerobic or aerobic. Accordingly, the present invention provides methods involving inhibiting the growth of, or killing of microbes via administration to a subject or contact with selective inhibitors of ATP production, as well as treating diseases resulting from microbial infection in a subject.

As used herein, a “patient” or “subject” refers to either a human or non-human animal. Non-human animals include any non-human animals capable of becoming infected with a microbe. Such non-human animals include vertebrates such as rodents, non-human primates, ovines, bovines, ruminants, lagomorphs, porcines, caprines, equines, canines, felines, ayes, piscines, and the like. In certain embodiments of the invention, the animals are mammals. Exemplary non-human mammals are porcines (e.g., pigs), murines (e.g., rats, mice, and lagomorphs (e.g., rabbits), and non-human primates (e.g., monkeys and apes).

As used herein, “treating” a disease in a subject or “treating” a subject having a disease refers to subjecting the subject to a pharmaceutical treatment, e.g., the administration of a selective inhibitor of the present invention, such that at least one symptom of the disease is decreased or prevented from worsening.

All microbes utilize at least one glycolytic pathway for energy generation. 3-bromopyruvate treatment can therefore be used to kill or prevent the growth of virtually any microbe. As used herein, microbes include microorganisms, such as bacteria, parasites and fungi. The methods described herein are particularly effective when the microbe is a bacteria.

There are several advantages of the inhibitory agents described herein over conventional antibacterial agents. First, inhibitors of the present invention are effective against all types of bacteria as all bacteria use sugar metabolism to live. Second, bacteria cannot develop resistant to the inhibitors of the present invention as resistance is based on MDR protein mediated drug resistance, the production and activity of which is driven by energy production. Further, there exists no other pathway the bacteria can resort to generate energy if the glycolytic pathway is blocked. Third, there are no toxic side effects to normal tissues. 3-bromopyruvate specifically targets bacterial cells. Since bacteria grow and replicate quickly, they consume great amounts of glycolytic substrates and so have transporters that transport these substrates. As 3-bromopyruvate is a lactate/pyruvate analog, it enters bacterial cells through these transporters to specifically target bacterial cells over normal cells, enhancing safety. As to any 3-bromopyruvate that may enter normal tissues, toxicity is reduced since normal tissues have the ability to survive from other forms of metabolism and contain sufficient antioxidants to neutralize 3-bromopyruvate that enters cells of the normal tissues.

As used herein, the phrase “selective inhibitor of ATP production” refers to any compound that is able to specifically modulate the activity of glyceraldehyde 3-phosphate dehydrogenase (GAPDH) or another enzyme that is limiting in the rapid production of ATP. For example, such metabolic pathways include the glycolytic pathway, oxidative phosphorylation pathway, and mitochondrial respiration.

Selective inhibitors of ATP production are represented by the general formula:

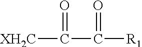

wherein X represents a halide, a sulfonate, a carboxylate, an alkoxide, or an amine oxide. In certain embodiments, X is a halide selected from the group consisting of: fluoride, bromide, chloride, and iodide. In one embodiment, the inhibitor is a 3-halopyruvate. In certain other embodiments, the 3-halopyruvate is selected from the group consisting of: 3-fluoropyruvate, 3-chloropyruvate, 3 -bromopyruvate and 3-iodopyruvate. In one embodiment, the 3-halopyruvate is 3-bromopyruvate. In other embodiments, X is a sulfonate selected from the group consisting of: triflate, mesylate and tosylate. In yet another embodiment, X is an amine oxide is dimethylamine oxide. In certain embodiments R₁ represents OR, H, N(R″)₂, C1-C6 alkyl, C6-C12 aryl, C1-C6 heteroalkyl, or a C6-C12 heteroaryl. Independently, in other embodiments, R″ represents H, C1-C6 alkyl, or C6-C12 aryl. Independently, in still other embodiments, R represents H, alkali metal, C1-C6 alkyl, C6-C12 aryl or C(O)R′; and R′ represents H, C1-C20 alkyl or C6-C12 aryl.

In an exemplary embodiment, the invention further provides inhibitors of ATP production represented by the general formula:

X—CH₂—CO—COOH,

wherein X represents a halide, a sulfonate, a carboxylate, an alkoxide, or an amine oxide. In certain embodiments, X is a halide selected from the group consisting of: fluoride, bromide, chloride, and iodide. In one embodiment, the inhibitor is a 3-halopyruvate. In certain embodiments, the 3-halopyruvate is selected from the group consisting of: 3-fluoropyruvate, 3-chloropyruvate, 3-bromopyruvate and 3-iodopyruvate. In one embodiment, the 3-halopyruvate is 3-bromopyruvate. In other embodiments, X is a sulfonate is selected from the group consisting of: triflate, mesylate and tosylate. In yet another embodiment, X is an amine oxide is dimethylamine oxide.

Other analogs, derivatives, prodrugs, metabolites and salts thereof of 3-bromopyruvate may also be used, provided that these compounds or compositions have an antibiotic effect. For example, when referring herein to a treatment using 3-bromopyruvate, it should be understood that the methods described herein may also be conducted with analogs, derivatives, prodrugs, metabolites and salts of 3-bromopyruvate, where applicable.

As described herein, 3-bromopyruvate is an especially functional antiglycolytic agent. 3-bromopyruvate inhibits glycolysis by binding and inhibiting GAPDH. Further, additional enzymes in the same pathway or those involved in ATP production may be targeted (i.e., glycolytic targets).

As used herein, the term “antibiotic” is used to refer to any molecule that prevents, inhibits or destroys life and as such, includes antibacterial agents, antifungicides, and antiparasitic agents.

In various aspects, the present invention provides a method of preventing or treating infections caused by a microbe, a method of killing a microbe, and methods of treating or preventing diseases or disorders resulting from microbial infection, as well as methods for reducing wound scarring and increasing wound healing. In particular, these methods are especially applicable when a microorganism is resistant to an antibiotic agent, by a mechanism, such as tolerance, inherent resistance, or acquired resistance. The methods of the present invention include administering a selective inhibitor of ATP production, such as 3-bromopyruvate, alone or in combination other antibiotic or chemotherapeutic agents to a subject.

As used herein, “inherent resistance” of a microorganism to an antibiotic agent refers to a natural resistance to the action of the agent even in the absence of prior exposure to the agent. As used herein, “acquired resistance” of a microorganism to an antibiotic agent refers to a resistance that is not inhibited by the normal achievable serum concentrations of a recommended antibiotic agent based on the recommended dosage. As used herein, “tolerance” of a microorganism to an antibiotic agent refers to when there is microstatic, rather than microcidal effect of the agent. Tolerance is measured by a ratio of the minimal bactericidal concentration (MBC) to the minimum inhibitory concentration (MIC) of greater than or equal to 32.

In various aspects, the present invention provides methods of preventing or treating microbial infection, fever, inflammation, sepsis in a subject in need thereof. The present invention further provides methods of preventing or reducing wound scarring and increased wound healing. The methods include administering to the subject an antiglycolytic halopyruvate, for example 3-bromopyruvate, thereby preventing or treating bacterial infection in the subject.

Typical infections that may be treated using the methods of the present invention include, but are not limited to bacterial meningitis, otitis media and externa, pneumonia, skin infections, eye infections, sinusitis, upper respiratory tract infections, gastritis, food poisoning, urinary tract infections, and sexually transmitted diseases. Typical infections that may be treated using the methods of the present invention include those resulting from infection from bacterial types including, but not limited to Streptococcus pneumoniae, Neisseria meningitides, Haemophilus influenzae, Streptococcus agalactiae, Listeria monocytogenes, Streptococcus aureus, mycoplasma pneumoniae, Clamydia pneumoniae, Legionella pneumophila, Mycobacterium tuberculosis, Staphylococcus aureus, Streptococcus pyogenes, Pseudomonas aeruginosa, Chlamydia trachomatis, Neisseria gonorrhoeae, Treponema pallidum, Ureaplasma urealyticum, Haemophilus ducreyi, Helicobacter pylori, Campylobacter jejuni, Salmonella, Shigella, Clostridium, Escherichia coli, Enterobacter, Klebsiella pneumoniae, Citrobacter freundii, Acinetobacter, Stenotrophomonas maltophilia, Serratia marcescens, Stenotrophomonas maltophilia, Bordetella pertussis, Brucella, Legionella, Moraxella catarrhalis, Yersinia, Neisseria, Staphylococcus saprophyticus, and the like.

Typical fungal infections that may be treated using the methods of the present invention include, but are not limited to aspergillosis, blastomycosis, coccidioidomycosis, cryptococcus, fungal sinusitis, histoplasmosis, mucormycosis, nail fungus, paracoccidioidomycosis, fungal pneumonia, sporotrichosis, valley fever and the like. Typical parasitic infections that may be treated using the methods of the present invention generally include, but are not limited to protozoa and parasitic flukes and worms. Those of ordinary skill in the art will appreciate that the invention provides means to target more than one microbe at a time, and so may be used to simultaneously target, for example, a bacterial and fungal infection, as commonly seen in conditions such as otitis externa.

One of skill in the art would understand that the selective inhibitors of the present invention may be formulated into a variety of pharmaceutical compositions suitable for different routes of administration. In various aspects, the selective inhibitors of the present invention may include a therapeutically-effective amount of one or more of the inhibitors, formulated together with one or more pharmaceutically acceptable carriers (additives) and/or diluents. In another aspect, certain embodiments, the selective inhibitors of the present invention may can be administered as such or in admixtures with pharmaceutically acceptable carriers and can also be administered in conjunction with other agents, such as antibiotics or chemotherapeutics. Conjunctive therapy thus includes sequential, simultaneous and separate, or co-administration of the active compound in a way that the therapeutic effects of the first administered one is not entirely disappeared when the subsequent is administered.

As described in detail below, the pharmaceutical compositions including the selective inhibitors of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. As such, administration may be by a number of different routes, such as by parenteral administration including intravenous injection, intraperitoneal injection or implantation, intramuscular injection or implantation, intrathecal injection, subcutaneous injection or implantation, intradermal injection, intraveneous injection, lavage, bladder wash-out, suppository, pessary, oral ingestion, topical application, enteric application, inhalation, aerosolization, nasal spray or drops, or ocular spray or drops. In a one embodiment, the pharmaceutical compositions are formulated for parenteral administration. In another embodiment, the pharmaceutical composition is formulated for intraarterial injection. In another embodiment, the pharmaceutical compositions are formulated for systemic administration.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of fowling pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and, magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 10 percent to about 30 percent. In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Aside from inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste. In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions of the invention may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in microencapsulated form, if appropriate, with one or more of the above-described excipients.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel. Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of the selective inhibitor, it is desirable to slow absorption from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations suitable for intraarterial administration including continuous intraarterial infusion include liposomes and polymers described herein. In one embodiment, the liposome is about 50 to 100 microns in diameter and is a pegylated stealth liposome with a PEG group attached to the outside of the lipid bilayer so as to avoid causing an immune response and escape degradation by the immune system. In another embodiment, the liposome is about 50 to 100 microns in diameter and is a nude liposome without the PEG group which may cause an immune response. In certain embodiment, the pegylated liposome may be used for systemic administration and the nude liposome may be used for local administration.

In another embodiment, the selective inhibitor of the present invention may be formulated in a polymer that is about 50 to 100 microns in diameter. In one embodiment, 3-bromopyruvate is attached to the polymer. In another embodiment, the granulocyte macrophage colony stimulating factor (GMCSF) is attached to the polymer. In another embodiment, the selective inhibitor of the present invention and GMCSF are attached to the polymer. In another embodiment, the polymer comprises selective inhibitor of the present invention and/or GMCSF. In certain embodiment, inert polymers may also be used as controls. Exemplary formulations comprising selective inhibitor of the present invention are determined based on various properties including, but not limited to chemical stability at body temperature, functional efficiency time of release, toxicity and optimal dose.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art. While it is possible for a compound of the present invention to be administered alone, it is preferable to administer the compound as a pharmaceutical formulation (composition).

In certain embodiments, the above-described pharmaceutical compositions comprise one or more of the inhibitors, a second chemotherapeutic agent, and optionally a pharmaceutically acceptable carrier. Alternatively, the selective inhibitor and chemotherapeutic agent may be administered separately. The term chemotherapeutic agent includes, without limitation, chemotherapeutic agent is selected from the group consisting of altretamine, asparaginase, BCG, bleomycin sulfate, busulfan, carboplatin, carmusine, chlorambucil, cisplatin, claladribine, 2-chlorodeoxyadenosine, cyclophosphamide, cytarabine, dacarbazine imidazole carboxamide, dactinomycin, daunorubicin-dunomycin, dexamethosone, doxurubicin, etoposide, floxuridine, fluorouracil, fiuoxymesterone, flutamide, fludarabine, goserelin, hydroxyurea, idarubicin HCL, ifosfamide, interferon alfa, interferon alfa 2a, interferon alfa 2b, interfereon alfa n3, irinotecan, leucovorin calcium, leuprolide, levamisole, lomustine, megestrol, melphalan, L-sarcosylin, melphalan hydrochloride, MESNA, mechlorethamine, methotrexate, mitomycin, mitoxantrone, mercaptopurine, paclitaxel, plicamycin, prednisone, procarbazine, streptozocin, tamoxifen, 6-thioguanine, thiotepa, vinblastine, vincristine, vinorelbine tartrate, or combinations thereof.

In any of the methods described herein, the selective inhibitor may be administered with, or the microbe contacted with the inhibitor and another agent, such as an antibiotic agent, including antibacterial agents, antifungal agents, antiparasitic agents. As such, in certain embodiments, the pharmaceutical compositions comprise one or more of the selective inhibitors, an antibiotic, and optionally a pharmaceutically acceptable carrier.

The combination of a selective inhibitor with an antibiotic agent, for which a microorganism is inherently resistant (e.g., the antibiotic has never been shown to be therapeutically effective against the organism in question), is used to overcome the resistance and confer susceptibility of the microorganism to the agent. Overcoming inherent resistance is especially useful for infections where the causative organism is becoming or has become resistant to most, if not all, of the currently prescribed antibiotics. Additionally, administering a combination therapy provides more options when toxicity of an antibiotic agent and/or price are a consideration.

Overcoming resistance can be conveniently measured in vitro. Resistance is overcome when the MIC for a particular antibiotic agent against a particular microorganism is decreased from the resistant range to the sensitive range. NCCLS standards are based on microbiological data in relation to pharmacokinetic data and clinical studies. Resistance is determined when the organism causing the infection is not inhibited by the normal achievable serum concentrations of the antibiotic agent based on recommended dosage. Susceptibility is determined when the organism responds to therapy with the antibiotic agent used at the recommended dosage for the type of infection and microorganism.

Acquired resistance in a microorganism that was previously sensitive to an antibiotic agent is generally due to mutational events in chromosomal DNA, acquisition of a resistance factor carried via plasmids or phage, or transposition of a resistance gene or genes from a plasmid or phage to chromosomal DNA. When a microorganism acquires resistance to an antibiotic, the combination of a selective inhibitor and antibiotic agent can restore activity of the antibiotic agent by overcoming the resistance mechanism of the organism. This is particularly useful for organisms that are difficult to treat or where current therapy is costly or toxic. The ability to use a less expensive or less toxic antibiotic agent, which had been effective in the past, is an improvement for certain current therapies. The re-introduction of an antibiotic agent would enable previous clinical studies and prescription data to be used in its evaluation. Activity is measured in vitro by MICs or kinetic kill curves and in vivo using animal and human clinical trials.

Enhanced activity of antiglycolytic agents of the invention may occur when the agent is combined with another antimicrobial in a manner which potentiates activity beyond the individual effects of the selective inhibitor or antibiotic agent alone or additive effects of peptide plus antibiotic agent. Enhanced activity is especially desirable in at least four scenarios: (1) the microorganism is sensitive to the antibiotic agent, but the dosage has associated problems; (2) the microorganism is tolerant to the antibiotic agent, and is inhibited from growing but is not killed; (3) the microorganism is inherently resistant to the antibiotic agent; and (4) the microorganism has acquired resistance to the antibiotic agent. Enhanced efficacy resulting from administration of the antibiotic agent in combination with a cationic peptide in the above scenarios: (1) allows for administration of lower dosages or antibiotic agent and selective inhibitor; (2) restores a cytocidal effect; (3) overcomes inherent resistance; and (4) overcomes acquired resistance.

Use of such a synergistic combination may permit a reduction in the dosage of one or both agents in order to achieve a similar therapeutic effect. This would allow smaller doses to be used, thus, decreasing the incidence of toxicity (e.g., from aminoglycosides) and lowering costs of expensive antibiotics (e.g., vancomycin). Alternatively, the antibiotic agent and selective inhibitor can be administered at therapeutic doses for each, but wherein the combination of the two agents provides even more potent effects.

Potentially useful antibacterial agents for co-administration include, but are not limited to, penicillins, cephalosporins, carbacephems, cephamycins, carbapenems, monobactams, aminoglycosides, glycopeptides, quinolones, tetracyclines, macrolides, and fluoroquinolones. Examples of antibacterial agents include, but are not limited to, Penicillin G (CAS Registry No. 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefinetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

Potentially useful antifungal agents for co-administration include, but are not limited to, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Potentially useful antiviral agents for co-administration include, but are not limited to, amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

Potentially useful antiparasitic agents for co-administration include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole (sulfamethoxazole/trimethoprim), and pentamidine isethionate.

The present invention further provides therapeutic method of preventing or treating microbial infection, inflammation, sepsis, and/or fever in a subject in need thereof. The method includes administering to the subject a halopyruvate, especially 3-bromopyruvate. A subject in need thereof may be a subject, e.g., a human, who has been diagnosed with or having a microbial infection, inflammation, sepsis, and/or fever or a subject who has been treated and has been, e.g., refractory to the previous treatment.

The present invention further provides a method of increasing wound healing or preventing or reducing wound scarring in a subject in need thereof. The method includes administering to the subject an antiglycolytic halopyruvate, especially 3-bromopyruvate. A subject in need thereof may be a subject, e.g., a human, who has an unhealed ailing wound, such as an open cut or sore.

The methods of the present invention may be used to treat a variety of types of microbial infections. In certain embodiments, the microbial infection is due to a microorganism having the capacity to perform one or more steps of glycolysis and preferably expresses GAPDH. For example, such microbes include all bacteria, parasites and fungi.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of agent required to prevent or treat a microbial infection a subject depends on many factors including the age and general health of the subject as well as the type and location of infection, the route of administration and the number of treatments to be administered. In view of these factors, the skilled artisan would adjust the particular dose as necessary.

Dosage may be based on the amount of the composition per kg body weight of the patient. For example, a range of amounts of compositions are contemplated, including about 0.001, 0.01, 0.1, 0.5, 1, 10, 15, 20, 25, 50 mg or more of such compositions per kg body weight of the patient. Other amounts will be known to those of skill in the art and readily determined.

In certain embodiments, the dosage of the subject compounds will generally be in the range of about 0.001 mg to about 10 mg per kg body weight, specifically in the range of about 0.1 mg to about 10 mg per kg, and more specifically in the range of about 0.1 mg to about 1 mg per kg. In one embodiment, the dosage is in the range of about 0.3 mg to about 0.6 mg per kg. In one embodiment, the dosage is in the range of about 0.4 mg to about 0.5 mg per kg.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms. The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained, hi addition, the combined use 3-bromopyruvate and a second agent, e.g., another chemotherapeutic agent or a scavenger compound, may reduce the required dosage for any individual component because the onset and duration of effect of the different components may be complimentary.

The term “effective amount” is defined as the amount of the compound or pharmaceutical composition that will elicit the biological or medical response of a tissue, system, animal or human that is being sought by the researcher, veterinarian, medical doctor or other clinician, e.g., reduction of bacterial count, alleviation of sepsis, inflammation and/or reduction in fever, increased rate of wound healing and decrease or prevention of wound scarring; and reduction of morbidity and/or mortality. For example, a “therapeutically effective amount” of, e.g., 3-bromopyruvate, with respect to the subject methods of treatment, refers to an amount of the compound in a preparation which, when applied as part of a desired dosage regimen brings about, e.g., a reduction in pathogenic bacterial count or killing of all pathogenic bacteria.

Although the invention has been described with reference to the above example, it will be understood that modifications and variations are encompassed within the spirit and scope of the invention. Accordingly, the invention is limited only by the following claims.

What is claimed is:

1. A method of preventing or treating microbial infection in a subject in need thereof comprising administering to the subject an antiglycolytic halopyruvate, thereby preventing or treating microbial infection in the subject.

2. The method of claim 1, wherein the antiglycolytic halopyruvate is 3-bromopyruvate.

3. The method of claim 1, wherein the 3-bromopyruvate is administered by intravenous injection, intraperitoneal injection or implantation, intramuscular injection or implantation, intrathecal injection, subcutaneous injection or implantation, intradermal injection, lavage, bladder wash-out, suppository, pessary, oral ingestion, topical application, enteric application, inhalation, aerosolization, nasal spray or drops, or ocular spray or drops.

4. The method of claim 1, further comprising administering an antibacterial agent.

5. The method of claim 1, further comprising administering an antifungal agent.

6. The method of claim 1, further comprising administering an antiviral agent.

7. The method of claim 1, further comprising administering an antiparasitic agent.

8. The method of claim 2, wherein the 3-bromopyruvate is administered in combination with a chemotherapeutic agent.

9. A method of preventing or treating inflammation resulting from microbial infection in a subject in need thereof comprising administering to the subject an antiglycolytic halopyruvate, thereby preventing or treating inflammation in the subject.

10. A method of preventing or treating sepsis resulting from microbial infection in a subject in need thereof comprising administering to the subject an antiglycolytic halopyruvate, thereby preventing or treating sepsis in the subject.

11. A method of preventing or treating fever resulting from microbial infection in a subject in need thereof comprising administering an antiglycolytic halopyruvate to the subject, thereby preventing or treating fever in the subject.

12. The method of claim 9, wherein the antiglycolytic halopyruvate is 3-bromopyruvate.

13. The method of claim 10, wherein the antiglycolytic halopyruvate is 3-bromopyruvate.

14. The method of claim 11, wherein the antiglycolytic halopyruvate is 3-bromopyruvate.

15. A method of increasing wound healing in a subject in need thereof comprising administering to the subject an antiglycolytic halopyruvate, thereby increasing wound healing in the subject.

16. A method of preventing or reducing wound scarring a subject in need thereof comprising administering to the subject an antiglycolytic halopyruvate, thereby preventing or reducing wound scarring in the subject.

17. The method of claim 15, wherein the antiglycolytic halopyruvate is 3-bromopyruvate.

18. The method of claim 16, wherein the antiglycolytic halopyruvate is 3-bromopyruvate.

19. A method of inhibiting the growth of a microbe comprising contacting the microbe with an antiglycolytic halopyruvate, thereby inhibiting growth of the microbe.

20. The method of claim 19, wherein the antiglycolytic halopyruvate is 3-bromopyruvate.
